(19) United States
(12) Patent Application Publication
(10) Pub. No.: US 2023/0068007 A1
(43) Pub. Date: Mar. 2, 2023

(54) SYSTEM AND METHOD FOR DISCOVERING DRUG ACTIVE SITE OF PROTEIN USING PATHOGENIC MUTATION

(71) Applicant: 3BILLON, Seoul (KR)

(72) Inventor: Jungsul LEE, Seoul (KR)

(73) Assignee: 3BILLON, Seoul (KR)

(21) Appl. No.: 17/822,899

(22) Filed: Aug. 29, 2022

(30) Foreign Application Priority Data
Aug. 30, 2021 (KR) 10-2021-0114968

(51) Int. Cl. G16B 15/30; G16B 20/30; G16B 30/00; G16B 15/20

(52) U.S. Cl. CPC G16B 15/30; G16B 20/30; G16B 30/00; G16B 15/20; C07K 2299/00

(57) ABSTRACT

Disclosed herein is a system for discovering a drug active site of protein using a pathogenic mutation. The system includes: a pathogenic mutation position detection unit for detecting a pathogenic mutation position corresponding to a pathogenic mutation in a three-dimensional structure of protein, using pathogenic mutation data containing information on a pathogenic mutation causing an abnormal protein function and protein structure data containing information on the three-dimensional structure corresponding to genetic sequencing of the protein; and a drug active site detection unit detecting a drug active site, corresponding to the pathogenic mutation position, and, to which a drug is bindable.

### CROSS-REFERENCE TO RELATED APPLICATIONS

The present application claims the benefit of Korean Patent Application No. 10-2021-0114968, filed Aug. 30, 2021, contents of which are incorporated herein by reference in its entirety.

### BACKGROUND

### 1. Technical Field

The present disclosure relates to a system and a method for discovering a drug active site of protein using a pathogenic mutation, and more specifically, to a system and a method for discovering a drug active site of protein using a pathogenic mutation, which can discover a drug active site capable of adjusting a protein function through drug-binding.

### 2. Background

Proteins may exist in a unique atypical form in three-dimensional space through interaction and folding between amino acids. Drugs for adjusting a protein function by activating protein or suppressing activity of the protein can have an effect only on some areas of a target protein. That is, the protein function can be adjusted at a specific site, and the remainder of the protein can have no effect on the protein function, even though compounds are bound to the remainder of the protein.

An active site of protein means a specific area on a three-dimensional structure of the protein. Namely, the active site of protein is referred to as a location in which the protein function is affected in a case in which compounds are bound to the corresponding site. The active site of protein is significantly important information in the virtual screening field for calculating and finding a binding force of compounds and proteins, using a plurality of compounds.

Binding energy between the protein and compounds can be calculated even if the compounds are located at any site on the three-dimensional space of the protein. However, there is a location which does not have any influence on the protein function even though the binding force is strong. It means that it is not possible to select compounds capable of adjusting the protein function, using just the binding energy or binding strength of compounds binding to the protein.

A precondition of the virtual screening is to know an active site of the target protein. When a user knows the active site, the user can select a compound with a high possibility to adjust the protein function. Therefore, finding the active site of the protein is essential for drug development. However, to date, a suitable method for finding an active site of a protein has not been suggested. So, drug development using an active site can be applied only to a minority of active sites having come to light by accident on an experimental basis.

### SUMMARY

The present disclosure has been made to solve the above-mentioned problems occurring in the prior art, and in an aspect of the present disclosure, it is an object to provide a system and a method for discovering a drug active site of protein using a pathogenic mutation, which can find a drug active site of protein through a three-dimensional structure analysis with respect to a pathogenic mutation position having a high possibility of adjusting a protein function.

To accomplish the above objects, in an aspect of the present disclosure, there is provided a system for discovering a drug active site of protein using a pathogenic mutation including: a pathogenic mutation position detection unit for detecting a pathogenic mutation position corresponding to a pathogenic mutation in a three-dimensional structure of protein, using pathogenic mutation data containing information on a pathogenic mutation causing an abnormal protein function and protein structure data containing information on the three-dimensional structure corresponding to genetic sequencing of the protein; and a drug active site detection unit detecting a drug active site, corresponding to the pathogenic mutation position, and, to which a drug is bindable.

In an embodiment of the present disclosure, the drug active site includes at least one among a structure directly exposed externally of the three-dimensional structure of the protein, and a structure exposed externally through a path connected externally of the three-dimensional structure.

In an embodiment of the present disclosure, the drug active site detection unit detects an empty space, adjacent to the pathogenic mutation position in a protein model formed in a three-dimensional structure corresponding to each atom contained in the three-dimensional structure of the protein, has a volume greater than or equal to a predetermined volume corresponding to the drug, and is connected externally of the protein model, as the drug active site.

In an embodiment of the present disclosure, the three-dimensional structure has a radius at a predetermined ratio of Van der Waals Radius of each atom.

In an embodiment of the present disclosure, a first coordinate included in the empty space is connected to a second coordinate located outside the protein model through a path spaced apart from the protein model.

To accomplish the above objects, in another aspect of the present disclosure, there is provided a method for discovering a drug active site of protein using a pathogenic mutation including: a pathogenic mutation position detecting operation of detecting a pathogenic mutation position corresponding to a pathogenic mutation in a three-dimensional structure of protein, using pathogenic mutation data containing information on a pathogenic mutation causing an abnormal protein function and protein structure data containing information on the three-dimensional structure corresponding to genetic sequencing of the protein; and a drug active site detecting operation of detecting a drug active site, corresponding to the pathogenic mutation position, and, to which a drug is bindable.

In an embodiment of the present disclosure, the drug active site includes at least one among a structure directly exposed externally of the three-dimensional structure of the protein, and a structure exposed externally through a path connected externally of the three-dimensional structure.

In an embodiment of the present disclosure, the drug active site detecting operation is to detect an empty space, which is adjacent to the pathogenic mutation position in a protein model formed in a three-dimensional structure corresponding to each atom contained in the three-dimensional structure of the protein, has a volume greater than or equal to a predetermined volume corresponding to the drug, and is connected externally of the protein model, as the drug active site.

In an embodiment of the present disclosure, the three-dimensional structure has a radius at a predetermined ratio of Van der Waals Radius of each atom.

In an embodiment of the present disclosure, a first coordinate included in the empty space is connected to a second coordinate located outside the protein model through a path spaced apart from the protein model.

### BRIEF DESCRIPTION OF THE DRAWINGS

FIG. 1 is a block diagram illustrating a system for discovering a drug active site of protein using a pathogenic mutation according to an embodiment of the present disclosure.

FIG. 2 is a flow chart illustrating a method for discovering a drug active site of protein using a pathogenic mutation according to an embodiment of the present disclosure.

FIG. 3 is a diagram illustrating a three-dimensional protein model of protein depicting a drug active site.

FIG. 4 is an enlarged view illustrating the drug active site of the three-dimensional protein model of FIG. 3.

FIG. 5 is an enlarged view illustrating a site, which cannot be proposed as the drug active site, of the three-dimensional protein model of FIG. 3.

FIG. 6 is an enlarged view illustrating a site, which cannot be proposed as the drug active site, of the three-dimensional protein model of FIG. 3.

### DETAILED DESCRIPTION

Hereinafter, the present disclosure will be described in more detail with reference to the accompanying drawings. The same reference numerals will be used for the same components in the drawings, and repeated descriptions of the same components will be omitted.

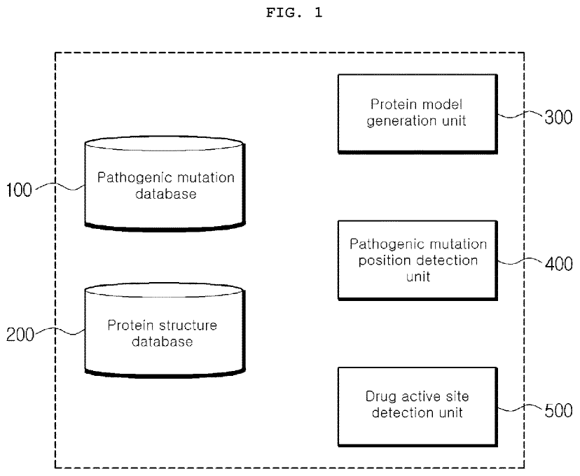

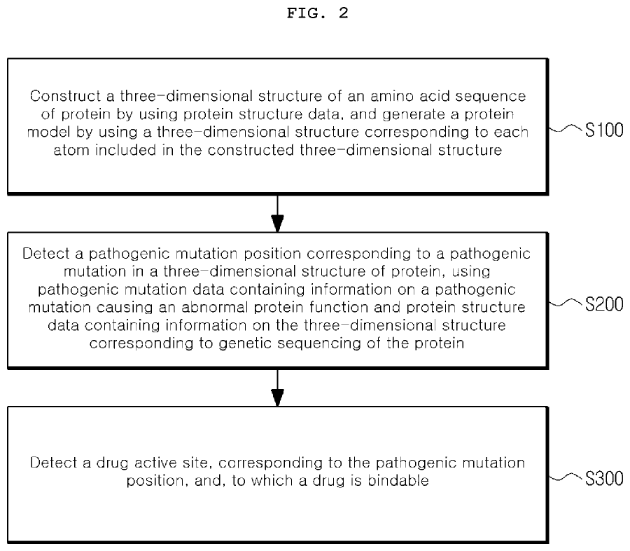

FIG. 1 is a block diagram illustrating a system for discovering a drug active site of protein using a pathogenic mutation according to an embodiment of the present disclosure, and FIG. 2 is a flow chart illustrating a method for discovering a drug active site of protein using a pathogenic mutation according to an embodiment of the present disclosure.

Referring to FIGS. 1 and 2, the system for discovering a drug active site of protein using a pathogenic mutation according to an embodiment of the present disclosure may include a pathogenic mutation database 100, a protein structure database 200, a protein model generation unit 300, a pathogenic mutation position detection unit 400, and a drug active site detection unit 500.

The pathogenic mutation database 100 stores pathogenic mutation data including information on a pathogenic mutation causing an abnormal protein function.

DNA contains genetic information of a living thing. A run of base sequences involved in expression of genetic traits among base sequences of the DNA is referred to as a gene, and a portion that is not involved in the expression of the genetic traits is referred to as non-coding DNA.

The gene can correspond to a base sequence area over a certain section of the DNA. The gene includes an exon section which contains information on protein and an intron section which does not contain information on protein and is involved in adjustment of expression.

The base sequence or nucleotide sequence refers to a sequence arrangement in which bases as components of nucleotide, which is a base unit of DNA or RNA of nucleic acid, are arranged in order.

The mutation (genetic mutation or base sequence mutation) refers to a sequence of a base or bases, which is different from the genetic base sequence of a normal human being, which is a comparison target, for instance, different in sequence, and may include substitution, addition or deletion of bases forming a sequence. Such substitution, addition or deletion of bases may be generated by various causes, for instance, structural differences including mutation, cleavage, deletion, duplication, reverse, or translocation of a chromosome.

The pathogenic mutation may mean a serious mutation as long as a disease can occur during the mutation. One person has an average of about five million mutations on the entirety of their genome, and has an average of about one hundred thousand mutations in the exon region expressed by the protein.

Most of these mutations do not or rarely have an influence on the living body, and very few mutations may cause severe symptoms. Some mutations which cause severe symptoms are referred to as pathogenic mutations.

Some pathogenic mutations are changed in the protein sequencing, and it may cause abnormal protein function. It is known that diseases are generated by the abnormal protein function. The pathogenicity of the mutation can be determined by direct experimental verification with respect to the abnormal protein function by the mutation and can be determined by distinguishing a very rare occurrence frequency of the corresponding mutation, patients who have similar symptoms, or repeated occurrence in the family line on the population from various angles. That is, the pathogenicity of the corresponding mutation is determined not only by the experimental verification but also by various additional criteria.

Occurrence of a pathogenic mutation causing a change in protein sequencing results in a loss or excessive activation of the protein function due to the change in protein sequence, and means that a disease associated therewith has occurred. Therefore, the change in protein sequence lets us know that a protein site in which the corresponding change occurs is a location which is important in the protein function, and such a location may be a protein active site.

In other words, non-pathogenic mutations among a number of mutations causing a change in the protein sequence are sites in which the protein function is maintained even if a portion of the protein sequence is changed due to the corresponding mutation. There is little probability that a drug combined with such a site, namely, the site maintaining the protein function even if a portion of the sequence is changed, adjusts the protein function.

On the other hand, since the protein site in which the pathogenic mutation has occurred seriously changes the protein function, there is a high possibility that a compound (e.g., drug) binding to the corresponding site, namely, the position of the pathogenic mutation, adjusts the protein function. Therefore, the protein site in which the pathogenic mutation has occurred can be proposed as a drug active site of the corresponding protein.

In an embodiment, the pathogenic mutation data includes at least one of kinds, positions, and occurrence frequencies of genetic mutations and pathogenic mutations corresponding to genes.

The pathogenic mutation data is established on the basis of a known gene mutation database, such as ClinVar, human gene mutation database (HGMD), Korean mutation database (KMD), online mendelian inheritance in man (OMIM), single nucleotide polymorphism database (dbSNP).

The protein structure database 200 stores protein structure data containing information on a three-dimensional structure corresponding to a sequence of genes or amino acids of proteins.

The protein structure data contains information on a three-dimensional structure formed through interaction and folding between amino acids of each protein.

In an embodiment, the protein structural data contains information on a three-dimensional structure of proteins predicted using a protein structure prediction method using artificial intelligence. For example, the method for predicting a protein structure using artificial intelligence can be implemented, using at least one of AlphaFold and RoseTTaFold.

The protein structure prediction method applied to the present disclosure can be implemented by the following known conventional art documents, and the detailed description related thereto can be omitted.

AlphaFold (Jumper, J., Evans, R., Pritzel, A. et al. Highly accurate protein structure prediction with AlphaFold. Nature (2021). https://doi.org/10.1038/s41586-021-03819-2), RoseTTaFold (Minkyung Baek, Frank DiMaio, et al. Accurate prediction of protein structures and interactions using a three-track neural network. Science (2021). https://doi.org/10.1126/science.abj8754).

The protein model generation unit 300 generates a protein model using the protein structure data. Specifically, the protein model generation unit 300 constructs a three-dimensional structure of an amino acid sequence of protein, using the protein structure data, and generates a protein model, using a three-dimensional structure corresponding to each atom included in the constructed three-dimensional structure (S100).

In an embodiment, the three-dimensional structure corresponding to each atom may include a sphere having a predetermined ratio of Van der Waals radius of each atom as a radius, but is not limited thereto, and may be constructed using one of polyhedrons of various structures. Accordingly, the protein model is defined in connection with the Van der Waals surface defined at the predetermined ratio of the Van der Waals Radius of each atom constituting protein.

In an embodiment, the predetermined ratio may include ½, but is not limited thereto, and various ratios can be set according to kinds of proteins, kinds of drugs to be combined, and kinds of compounds to be combined.

The pathogenic mutation position detection unit 400 detects a pathogenic mutation position corresponding to the pathogenic mutation in the three-dimensional structure of the protein, using the pathogenic mutation data and the protein structure data. The pathogenic mutation data contains information on the pathogenic mutation causing abnormal protein function. The protein structure data contains information on a three-dimensional structure corresponding to the protein genetic sequencing. The three-dimensional structure of the protein includes a three-dimensional structure of the amino acid sequencing of the protein (S200).

In an embodiment, the pathogenic mutation position detection unit 400 detects a pathogenic mutation position corresponding to a base sequence position of the pathogenic mutation in the three-dimensional structure of the protein formed, using the protein structure data.

In an embodiment, the pathogenic mutation position detection unit 400 detects a pathogenic mutation position corresponding to the base sequence position of the pathogenic mutation in the protein model formed by the protein model generation unit 300.

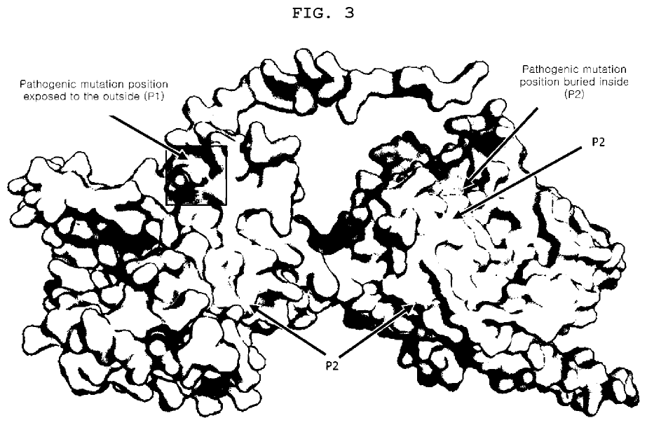

FIG. 3 is a diagram illustrating a three-dimensional protein model of protein depicting a drug active site.

The protein illustrated in FIG. 3 is a protein expressed by a SLC26A4 gene which causes an auditory damage or a thyroid disorder.

The amino acid sequence of the SLC26A4 gene is as follows, wherein portions indicated by bold letters, underlines, ‘<<’, and ‘>>’ are mutation positions known as the pathogenic mutation:

. . . MAAPGGRSEPPQLPEYSCSYMVSRPVYSELAFQQQHERRL
QERKTLRESLAKCCSCSRKRAFGVLKTLVPILEWLPKYRVKEWLLS
DVI<<S>>GVSTGLVATLQGMAYALLAAVPVGYGLYSAFFPILTYF
IFGTSRHISVGPFPVVSLMVGSVVLSMAPDEHFLVSSSNGTVLNTT
MIDTAARDTARVLIASALTLLVGIIQLIFGGLQIGFIVRYLADPLV
GGFTTAAAFQVLVSQLKIVLNVSTKNYNGVLSIIYTLVEIFQNIGD
TNLADFTAGLLTIVVCMAVKELNDRFRHKIPVPIPIEVIVTIIATA
ISYGANLEKNYNAGIVKSIPRGFLPPELPPVSLFSEMLAASFSIAV
VAYAIAVSVGKVYATKYDYTIDGNQEFIAFGISNIFSGFFSCFVAT
TALSR<<T>>AVQESTGGKTQVAGIISAAIVMIAILALGKLLEPLQ
KSVLAAVVIANLKGMFMQLCDIPRLWRQNKIDAVIWVFTCIVSIIL
GLDL<<G>>LLAGLIFGLLTWLRVQFPSWNGLGSIPSTDIYKSTKN
YKNIEEPQGVKILRFSSPIFYGNVDGFKKCIKSTVGFDAIRVYNKR
LKALRKIQKLIKSGQLRATKNGIISDAVSTNNAFEPDEDIEDLEEL
DIPTKEIEIQVDWNSELPVKVNVPKVPIHSLVLDCGAISFLDVVGV
RS<<L>>RVIVKEFQRIDVNVYFASLQDYVIEKLEQCGFFDDNIRK
DTFFLTV<<H>>DAILYLQNQVKSQEGQGSILETITLIQDCKDTLE
LIETELTEEELDVQDEAMRTLAS . . .

Referring to FIG. 3, the pathogenic mutant position detection unit 400 detects five pathogenic mutation positions P1 and P2 from the SLC26A4 protein.

In an embodiment, the pathogenic variant position detection unit 400 constructs a three-dimensional structure for the amino acid sequence of the SLC26A4 gene, using the protein structural data, and specifies and detects pathogenic mutation positions of <<S>>, <<T>>, <<G>>, <<L>>, and <<H>>, which are pathogenic mutations of the SLC26A4 gene obtained, using the pathogenic mutation data in the constructed three-dimensional structure.

In an embodiment, the pathogenic mutation position detection unit 400 specifies and detects pathogenic mutation positions P1 and P2 of <<S>>, <<T>>, <<G>>, <<L>>, and <<H>>, which are pathogenic mutations of the SLC26A4 gene in the protein model of the SLC26A4 gene generated by the protein model generation unit 300.

The pathogenic mutation positions are divided into a pathogenic mutation position P1 exposed externally, namely, to the surface of the protein structure, and a pathogenic mutation location P2 buried inside, namely, located inside the protein structure.

The drug active site detection unit 500 detects a drug active site, corresponding to the pathogenic mutation positions P1 and P2 based on the binding energy between the protein and the drug, and, to which a drug is bindable (S300).

The drug active site detection unit 500 selects a position exposed to the surface of the protein model among the pathogenic mutation positions P1 and P2 as a drug active site so that the drug can bind to the protein.

The drug active site detection unit 500 selects a position partially surrounded by a peripheral structure among the pathogenic mutation positions P1 and P2 as a drug active site so as to facilitate maintenance of the binding state.

In an embodiment, the drug active site includes at least one among a structure directly exposed externally of the three-dimensional structure of the protein, namely, the protein model, and a structure exposed externally through a path connected externally of the three-dimensional structure of the protein, namely, the protein model.

In other words, the drug active site includes a structure which is directly exposed to the surface of the three-dimensional protein structure, a structure which is exposed to the surface of the three-dimensional protein structure but is hollowed inwardly, or a structure accessible through a path like a cave.

The drug active site detection unit 500 detects an empty space satisfying a predetermined condition in the protein model as a drug active site. The empty space of the protein model is defined as a space formed outside of the three-dimensional structure based on the surface of the protein model.

The empty space satisfying the predetermined condition is adjacent to the pathogenic mutation position (first condition), and has a volume in which the compound can exist (second condition), and includes an empty space connected externally of the protein model (third condition).

The drug active site detection unit 500 determines the first condition on the basis of a distance to the pathogenic mutation position.

The drug active site detection unit 500 determines the second condition by comparing that the volume of the empty space is larger than or equal to a predetermined volume corresponding to the drug. Specifically, the drug active site detection unit 500 arranges virtual unit spheres corresponding to the drug in the empty space, and determines the second condition, using at least one among the arranged virtual unit spheres. For example, the volume of the empty space is calculated, using at least one among the number of the virtual unit spheres arranged in the empty space or the arrangement structure, and the calculated volume of the empty space is compared with the predetermined volume so as to determine the second condition.

The predetermined volume includes a predetermined value depending on the kinds of drugs or compounds to be bound to the protein. For example, the predetermined volume is larger than or equal to 300 angstrom³, but is not limited thereto.

The drug active site detection unit 500 determines the third condition by determining whether or not a first coordinate included in the empty space is connected to a second coordinate located outside the protein model through a path spaced apart from the protein model.

In other words, the first coordinate included in the empty space is connected to the second coordinate located outside the protein model through the path spaced apart from the protein model.

Here, the path spaced apart from the protein model includes a passage connecting the first coordinate and the second coordinate without touching or overlapping the three-dimensional structure (e.g., a sphere) having the predetermined ratio of Van der Waals Radius of each atom contained in the three-dimensional structure of the protein.

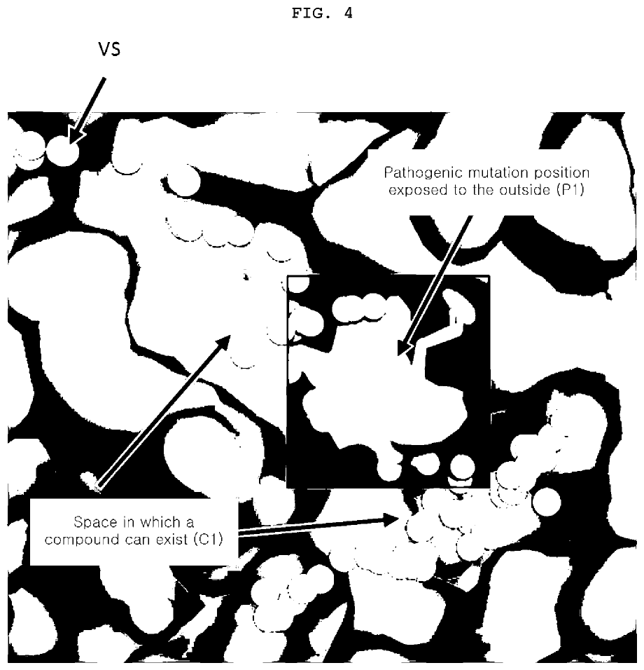

FIG. 4 is an enlarged view illustrating the drug active site of the three-dimensional protein model of FIG. 3.

Referring to FIG. 4, an empty space C1 in which a compound exists (i.e., binds) to the pathogenic mutation position P1 exposed externally, among the five pathogenic mutant positions P1 and P2, is detected.

Referring to FIGS. 3 to 6, the white surface of the protein model refers to the pathogenic mutation position P1 exposed externally, and the bar-shaped structure refers to the pathogenic mutation position P2 buried inside.

The drug active site detection unit 500 measures a volume by arranging the virtual unit spheres VS with respect to the empty space C1 corresponding to the pathogenic mutation position P1 exposed externally, and compares the measured volume with the predetermined volume corresponding to the drug.

The drug active site detection unit 500 determines the empty space C1, which is adjacent to the pathogenic mutation position P1, has the measured volume larger than or equal to the predetermined volume corresponding to the drug, and is connected externally of the protein model corresponding to the pathogenic mutation position exposed externally, as a drug active site.

It has been determined that the pathogenic mutation corresponding to the empty space C1 is a mutation having pathogenicity great enough to cause dysacusis in a case in which amino acids are changed. So, in a case in which the protein structure corresponding to the empty space C1 is changed, it is predicted to have a great influence on the protein function. Therefore, the empty space C1 may be a position having a high possibility of a drug active site, and may increase the probability of new drug discovery in a case in which a drug discovery is performed around the corresponding site.

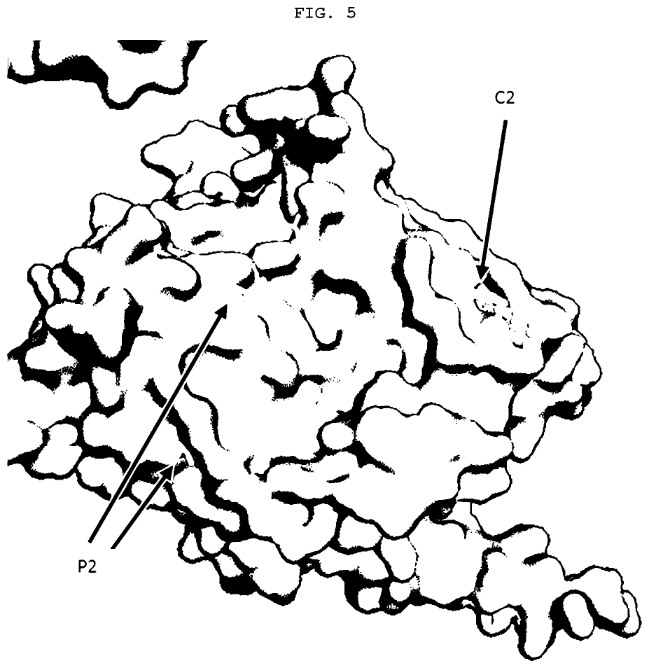

FIG. 5 is an enlarged view illustrating sites, which cannot be proposed as the drug active site, of the three-dimensional protein model of FIG. 3.

Referring to FIG. 5, in case of performing drug screening using a binding force between a compound (drug) and protein, the protein model includes an empty space C2 which can be determined to have a sufficient binding force between lots of drugs and the protein since having the inwardly hollowed structure.

The empty space C2 has a volume in which the compound can exist (satisfying the second condition) and is connected externally of the protein model (satisfying the third condition), but there is no pathogenic mutation at a close distance (not satisfying the first condition). So, it is expected that there will be little influence on the protein function even if the protein structure is changed at the corresponding position. Therefore, the empty space C2 cannot be proposed as a drug active site.

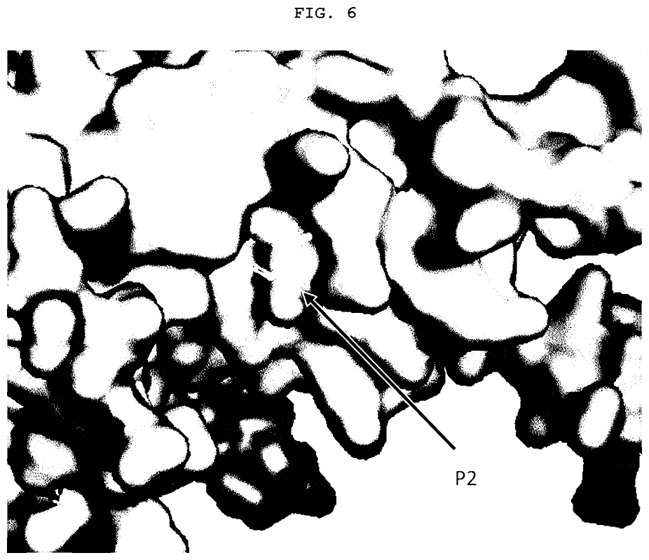

FIG. 6 is an enlarged view illustrating a site, which cannot be proposed as the drug active site, of the three-dimensional protein model of FIG. 3.

Referring to FIG. 6, the protein model includes the pathogenic mutation location P2 to which a compound, such as a drug, is not accessible from the outside due to existing inside.

The pathogenic mutation position P2 existing in the protein model satisfies the condition to be adjacent to the pathogenic mutation position (satisfying the first condition), but has no empty space which is located inside and in which the compound exists (not satisfying the second condition) and has no path or route connected to the empty space, which exists inside, from the outside even if there is the empty space inside. So, the drug is not accessible to the pathogenic mutation position P2. Therefore, the pathogenic mutation position P2 existing in the protein model cannot be proposed as a drug active site.

While the exemplary embodiments of the present disclosure have been described in more detail with reference to the accompanying drawings, it will be understood by those of ordinary skill in the art that various modifications, changes and equivalents may be made without deviating from the spirit or scope of the disclosure described in the following claims.

### Advantageous Effects

The system and method for discovering a drug active site of protein using a pathogenic mutation according to the present disclosure can discover a drug active site with respect to protein known in information on a pathogenic mutation.

In addition, drug discovery can be performed focused on the discovered drug active site to improve the probability of new drug development.

1. A system for discovering a drug active site of protein using a pathogenic mutation comprising:
a pathogenic mutation position detection unit for detecting a pathogenic mutation position corresponding to a pathogenic mutation in a three-dimensional structure of protein, using pathogenic mutation data containing information on a pathogenic mutation causing an abnormal protein function and protein structure data containing information on the three-dimensional structure corresponding to genetic sequencing of the protein; and
a drug active site detection unit detecting a drug active site, corresponding to the pathogenic mutation position, and, to which a drug is bindable.

2. The system according to claim 1, wherein the drug active site includes at least one among a structure directly exposed externally of the three-dimensional structure of the protein, and a structure exposed externally through a path connected externally of the three-dimensional structure.

3. The system according to claim 1, wherein the drug active site detection unit detects an empty space, which is adjacent to the pathogenic mutation position in a protein model formed in a three-dimensional structure corresponding to each atom contained in the three-dimensional structure of the protein, has a volume greater than or equal to a predetermined volume corresponding to the drug, and is connected externally of the protein model, as the drug active site.

4. The system according to claim 3, wherein the three-dimensional structure has a radius at a predetermined ratio of Van der Waals Radius of each atom.

5. The system according to claim 3, wherein a first coordinate included in the empty space is connected to a second coordinate located outside the protein model through a path spaced apart from the protein model.

6. A method for discovering a drug active site of protein using a pathogenic mutation comprising:
a pathogenic mutation position detecting operation of detecting a pathogenic mutation position corresponding to a pathogenic mutation in a three-dimensional structure of protein, using pathogenic mutation data containing information on a pathogenic mutation causing an abnormal protein function and protein structure data containing information on the three-dimensional structure corresponding to genetic sequencing of the protein; and
a drug active site detecting operation of detecting a drug active site, corresponding to the pathogenic mutation position, and, to which a drug is bindable.

7. The method according to claim 6, wherein the drug active site includes at least one among a structure directly exposed externally of the three-dimensional structure of the protein, and a structure exposed externally through a path connected externally of the three-dimensional structure.

8. The method according to claim 6, wherein the drug active site detecting operation is to detect an empty space, which is adjacent to the pathogenic mutation position in a protein model formed in a three-dimensional structure corresponding to each atom contained in the three-dimensional structure of the protein, has a volume greater than or equal to a predetermined volume corresponding to the drug, and is connected externally of the protein model, as the drug active site.

9. The method according to claim 8, wherein the three-dimensional structure has a radius at a predetermined ratio of Van der Waals Radius of each atom.

10. The method according to claim 8, wherein a first coordinate included in the empty space is connected to a second coordinate located outside the protein model through a path spaced apart from the protein model.
